(19) United States
(12) Patent Application Publication
(10) Pub. No.: US 2021/0375402 A1
(43) Pub. Date: Dec. 2, 2021

(54) DOUBLE-LAYER NEURAL NETWORK ALGORITHM FOR HIGH-PRECISION ENERGY CALCULATION OF ORGANIC MOLECULAR CRYSTAL STRUCTURE

(71) Applicant: SHENZHEN JINGTAI TECHNOLOGY CO., LTD., Guangdong (CN)

(72) Inventors: Yingdi JIN, Guangdong (CN); Peiyu ZHANG, Guangdong (CN); Qun ZENG, Guangdong (CN); Guangxu SUN, Guangdong (CN); Lipeng LAI, Guangdong (CN); Jian MA, Guangdong (CN); Shuhao WEN, Guangdong (CN)

(73) Assignee: SHENZHEN JINGTAI TECHNOLOGY CO., LTD., Guangdong (CN)

(21) Appl. No.: 16/960,027

(22) PCT Filed: Sep. 5, 2019
(86) PCT No.: PCT/CN2019/104545

§ 371 Date: Jul. 3, 2020

(30) Foreign Application Priority Data
Jul. 24, 2019 (CN) 201910671195.5

(51) Int. Cl. G16C 20/70; G16C 20/30; G06N 3/04

(52) U.S. Cl. CPC G16C 20/70; G06N 3/0454; G16C 20/30

(57) ABSTRACT

The invention pertains to the field of organic molecular crystal structure prediction, and particularly related to a double-layer neural network algorithm for high-precision energy calculation of organic molecular crystal structure, including the first round of conventional crystal structure prediction; extract all molecular conformations from existing crystals and calculate their energies; extract all molecular dimers within the Van der Waals radius of the central unit cell and calculate the intermolecular interaction energies; perform molecular conformation analysis to build a convolutional neural network of single-molecule conformational energies; build a molecular dimer energy-corrected convolutional neural network; calculate the total crystal energies. The invention improves the accuracy of energy calculation in the process of predicting the crystal structure of drug molecules while maintaining the calculation speed; fast and accurate energy calculation will guide the CSP process to quickly find a truly stable crystal form on the correct potential energy surface.

### BACKGROUND

### Technical Field

The invention pertains to the field of organic molecular crystal structure prediction, and particularly applied to a double-layer neural network algorithm for high-precision energy calculation of organic molecular crystal structure.

### Description of Related Art

The chemical compound's characteristic of forming different crystal structures is called polymorphism. The key physical and chemical properties of the compound, such as density, morphology, solubility, and dissolution rate, are strongly affected by its crystal form. For drugs, the crystal form can strongly affect the bioavailability of the drug and ultimately affect the drug's therapeutic performance. Experimental polymorphic drug screening has become an indispensable part of the standard drug development process. In the experiment, people set the key crystallization parameters manually or with the help of a robot, but the correct crystallization conditions are difficult to obtain in a short time through the experiment. An alternative is to use computer simulation for crystal structure prediction (CSP) of drug molecules, to find a variety of potential stable crystal forms, and then focus experiments on a few potential crystal forms with clear targets.

In the past decade, both inorganic and organic crystal prediction (CSP) have made great progress. Despite many similarities, the prediction of inorganic and organic crystals needs to face very different challenges. In inorganic CSP, people are concerned about the opening and closing of chemical bonds and electronic properties, while organic CSP is more concerned about structural transition and phase transition. Drug development is related to the CSP of organic molecules. There are currently two major challenges in this field, one is the completeness of the spatial sampling of the crystal, and the other is the accuracy of the final energy ranking of the crystal structure.

For the first challenge, the completeness of crystal space sampling, is usually completed through a large-scale crystal structure search. In this process, a large number of crystal structures will be generated, requiring a large amount of energy calculations. For inorganic CSP, the crystal energy is usually obtained directly using the calculation method of quantum mechanical accuracy. But due to the too complicated system and too high chemical space dimension of organic molecular crystal, there are too many crystal structures that requires energy calculation in the organic CSP which prevents the application of calculation methods that directly use quantum mechanical accuracy in organic CSP. An alternative method is to use the classical mechanics method with low accuracy and fast calculation speed; but due to its accuracy limitation, the potential energy surface description of structural prediction is usually inaccurate.

Accurate calculation of the small energy difference between different low-energy crystal structures requires high-precision quantum mechanical calculations, and the time complexity of high-precision quantum mechanical calculations is O (N³)˜O (N⁴) of the electron number N in the system. When the system increases, the energy calculation of a large number of crystal structures generated during the CSP process with the quantum mechanical accuracy becomes the bottleneck of CSP. One solution is to introduce machine learning algorithms for energy correction, while basically maintaining the calculation speed of classical mechanics, and improving the energy calculation accuracy to quantum mechanical accuracy.

### SUMMARY

In view of the above technical problems, the present invention uses machine learning technology to provide a process for performing rapid and high-precision energy calculations on a large number of crystal structures generated during the prediction of organic molecular crystal structures to improve the efficiency and accuracy of crystal structure energy calculations. In order to achieve the above purpose, based on the double-layer deep convolutional neural network of periodic crystals and a large number of existing crystal structures and their energy data, a high-precision energy calculation method suitable for organic molecular crystals is designed. The framework designed by this method can be applied to any first-principles calculation method and semi-empirical algorithm.

The technical solutions adopted are the double-layer neural network algorithm for high-precision energy calculation of organic molecular crystal structure includes the following steps:

(1) Run a Conventional Crystal Structure Prediction

After energy ranking, determine a cut-off value of relative energy E₀; take out all crystal structures with relative energy lower than the cut-off value to get a set of crystal structures, and marked as {Sᵢ}, subscript i means to all crystal structures whose energy is lower than the cut-off value; calculate the energies of the structures in the set with quantum mechanical accuracy to obtain an accurate energies set as {Eᵢ}.

(2) Extract Molecular Conformations and Calculate their Energies

Extract all molecular conformations from the crystal structure set{Sᵢ}, mark the molecular conformation set as {Cₐ}, a means all molecular conformations that have occurred in all crystal structures; calculate the energies of the conformations in the set with quantum mechanical accuracy to get the accurate energies set as {Eₐ^{mol}}.

(3) Extract Molecular Dimers and Calculate Intermolecular Interaction Energy

Select a central unit cell for a crystal from the crystal structures set{Sᵢ}, and take a circle of molecules within the range of Van der Waals force for all molecules in the central unit cell. The range of Van der Waals force is defined as at least the distance between one pair atoms in two molecules is less than the sum of Van der Waals radius of the two atoms plus 1.5 Å; Extract the central unit cell and all molecular dimers {D_{AB}} within its Van der Waals force range, and calculate the intermolecular interaction energy in each dimer with quantum mechanical accuracy, the formula is as shown below:

E_{AB_inter_QM}=E_{AB_tot_QM}−E_{A_QM}−E_{B_QM}

E_{AB_inter_QM} is the intermolecular interaction energy in the dimer AB, E_{AB_tot_QM} is the total energy in the dimer, E_{A_QM} is the energy of the molecule A in the dimer, and similarly E_{B_QM} represents the energy of the molecule B in the dimer, all the energies are calculated with quantum mechanics accuracy.

(4) Build a Convolutional Neural Network of Single Molecule Conformational Energy

Mark the molecular flexible dihedral angles set as {Aₗ}, l means all the flexible dihedral angles in the molecules; set a series of fixed angle values as {θₛ} for one of the angles Aₗ; perform energy-constrained optimization calculations with the quantum mechanical accuracy to obtain a batch of molecular conformations and energies; build a convolutional neural network. The atomic distance matrix Mₗ in the molecule is used as an input of the neural network, and the molecular conformational energy as an output. Use this batch of molecular conformations and the interatomic distance matrices of all the conformations obtained in step (2), and their conformation energies to train the parameters of the neural network.

(5) Build a Molecular Dimer Energy-Corrected Convolutional Neural Network

Calculate the intermolecular interaction energies in all dimers obtained in step (3) with the classical mechanical accuracy; calculate the difference of intermolecular interaction energy in the dimer between the quantum mechanical accuracy and the molecular mechanical accuracy:

ΔE_{AB_inter}=E_{AB_inter_QM}−E_{AB_inter_MM}

wherein E_{AB_inter_QM} is the intermolecular interaction energy in the dimer calculated with quantum mechanical accuracy which is calculated in step (3), and E_{AB_inter_MM} is the intermolecular interaction energy in the dimer calculated with classical mechanical accuracy.

Build up interatomic distance matrices of dimer set{D_{AB}}; build a convolutional neural network wherein the interatomic distance matrix in the dimer as the input of the neural network, and the high-precision interaction correction of the dimer as the output; use the interatomic distance matrices {M_{AB}} of the dimers {D_{AB}} and the modified values {ΔE_{AB_inter}} of their interaction energies to train the parameters of the neural network;

(6) Calculate Crystal Energy

Calculate the total energy for any crystal structure S generated during the crystal prediction process:

$${E}_{S} = {\sum }_{a}^{mols} {E}_{a} + {\sum }_{AB}^{dimers} {E}_{AB\_MM} + {\sum }_{AB}^{dimers} Δ {E}_{AB\_inter} + \sum {E}_{others \_MM}$$

Here Σₐ^{mols} Eₐ is the sum of all intramolecular energies; Σ_{AB}^{dimers}E_{AB_MM} is the sum of all dimer energies calculated with classical mechanical accuracy, and Σ_{AB}^{dimers}ΔE_{AB_inter} is the sum of the correction amounts of the intermolecular interaction energies in all dimmers calculated by the neural network in step (5); ΣE_{others_MM} is all remaining interactions calculated by conventional classical mechanics.

The double-layer neural network algorithm for high-precision energy calculation of organic molecular crystal provided by the present invention has the following technical effects:

(1) The accuracy of energy calculation during the prediction of the crystal structure of drug molecules has been improved, and the accuracy of energy calculation of crystal structure has been improved from classical mechanical accuracy to quantum mechanical accuracy;

(2) The accuracy of the optimization algorithm direction in the crystal structure prediction process is improved, and the high-precision energy will guide the CSP to quickly find the truly stable crystal form on the correct potential energy surface.

### BRIEF DESCRIPTION OF THE DRAWINGS

FIG. 1(a) shows one of the two different crystal forms of the same molecule in the embodiment;

FIG. 1(b) shows the molecular conformation extracted from the crystal in FIG. 1(a), which indicates that the same molecule would have different conformations when forming the crystal;

FIG. 1(c) shows the second one of the two different crystal forms of the same molecule in the embodiment;

FIG. 1(d) shows the molecular conformation extracted from the corresponding crystal in FIG. 1(c), which indicates that the same molecule will have different conformations when forming the crystal;

FIG. 2(a) shows dimer1 and dimer2 representing the two dimers present in the crystal Sj;

FIG. 2(b) shows that the dimer's judgment condition is that when the distance between the two nearest atoms in two molecules is less than the sum of the Van der Waals radius of the two atoms plus 1.5 Å, the two molecules are judged to form a dimer.

### DESCRIPTION OF THE EMBODIMENTS

The specific technical solutions of the present invention will be described with the embodiments.

The high-precision energy calculation method used in organic molecular crystal structure prediction includes the following steps:

(1) Run the First Round of Conventional Crystal Structure Prediction

After a round of conventional crystal structure prediction, the energy cutoff value E₀ is determined after standard energy ranking with quantum mechanical accuracy. All crystal structures with relative energy lower than the cutoff value E₀ are taken out as the crystal structure set {Sᵢ} and its quantum mechanical accuracy energy set as {Eᵢ}.

(2) Extract Molecular Conformation and Calculate its Energy

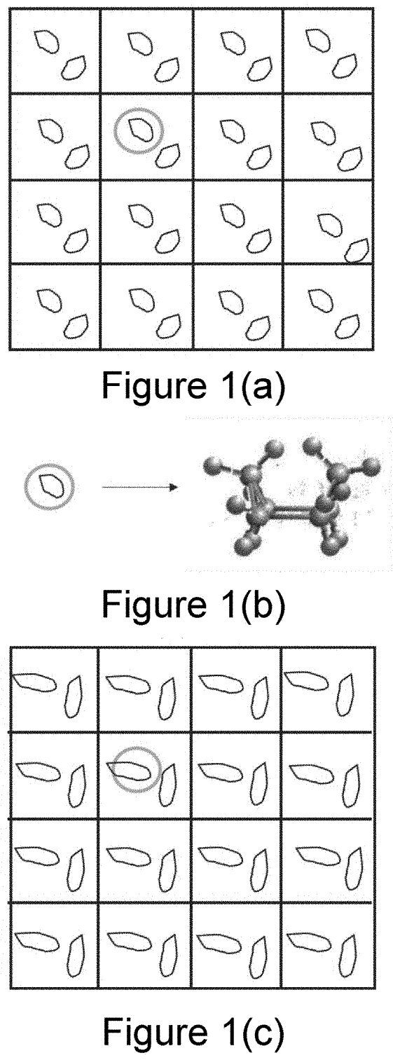

As shown in FIG. 1(b) and FIG. 1(d), molecules with the same chemical formula can have different conformations when forming crystals, that is, the flexible dihedral angle of the molecule can be rotated at different angles. FIG. 1(a) and FIG. 1(c) are two different crystal forms of the same molecule. The schematic diagrams of the two molecules in FIG. 1(b) and FIG. 1(d) show that when the same molecule forms a crystal, there would be different conformations;

Thus, in this step, the molecular conformation set extracted from the crystal structure set {Sᵢ} is marked as {Cₐ}, a means all the molecular conformations that have occurred in all crystal structures and hereinafter means the same. Calculate the energies of the conformations in the set with the quantum mechanical accuracy to get the accurate energy set as {Eₐ^{mol}}.

(3) Extract Molecular Dimers and Calculate the Intermolecular Interaction Energy

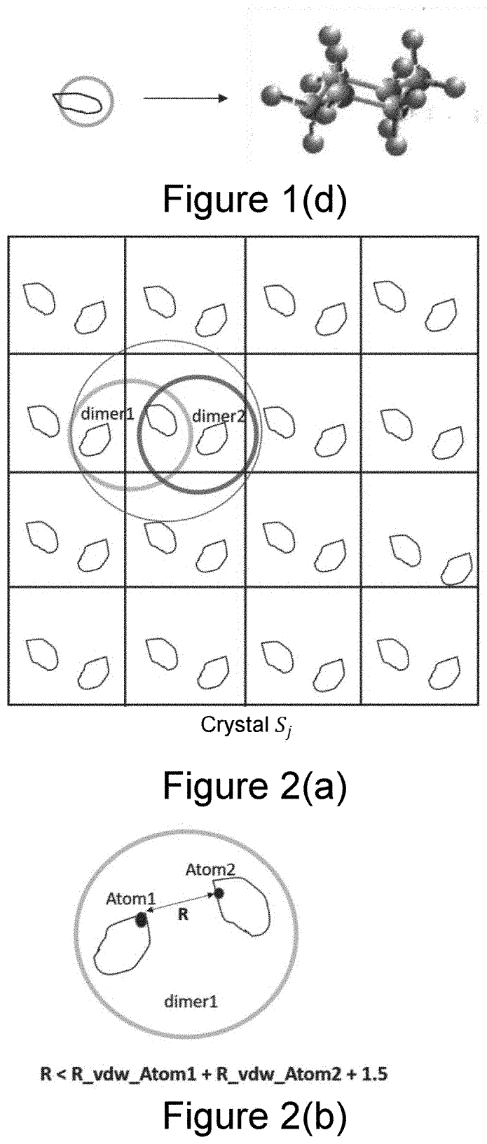

As shown in FIG. 2(a), dimer1 and dimer2 respectively represent two dimers in the crystal, and FIG. 2(b) indicates that the dimer's judgment condition is that when the distance of the two atoms of the two molecules with the closest distance is less than the sum of Van der Waals radius of the two atoms plus 1.5 Å, the two molecules are judged to form a dimer.

Select a central unit cell for a crystal Sᵢ from the crystal structures set {Sᵢ}, and take a circle of molecules within their Van der Waals force range for all molecules in the central unit cell; the range of Van der Waals force is defined as at least the distance between one pair atoms in two molecules (As shown in FIG. 2(b) the distance R between atom1 and atom2) is less than the sum of Van der Waals radius of the two atoms plus 1.5 Å;

Extract molecules from the central unit cell and all molecular dimers {D_{AB}} (as shown in FIG. 2(a) dimer1 and dimer2) within their Van der Waals force range, and calculate the intermolecular interaction energy in each dimer with quantum mechanical accuracy, the formula is as:

E_{AB_inter_QM}=E_{AB_tot_QM}−E_{A_QM}−E_{B_QM}

E_{AB_inter_QM}is the intermolecular interaction energy in the dimer AB, E_{AB_tot_QM}is the total energy in the dimer, E_{A_QM} is the energy of the molecule A in the dimer, and similarly E_{B_QM} represents the energy of the molecule B in the dimer, all the energies are calculated with quantum mechanical accuracy.

(4) Build Convolutional Neural Network of Single Molecule Conformational Energy

Mark the molecular flexible dihedral angle set as {Aₗ}, l means all the flexible dihedral angles in the molecules; set a series of fixed angle values as {θₛ} for one of the angles Aₗ, and perform energy-constrained optimization calculations with the quantum mechanical accuracy to obtain a batch of molecular conformations and energies; Build a convolutional neural network, the atomic distance matrix Mₗ in the molecule is used as the input of the neural network, and the molecular conformational energy as the output; and use this batch of molecular conformations and the interatomic distance matrices of all the conformations obtained in step (2), and their conformation energies to train the parameters of the neural network.

(5) Build Molecular Dimer Energy-Corrected Convolutional Neural Network

Calculate the intermolecular interaction energy in all dimers obtained in step (3) with the classical mechanical accuracy; Calculate the intermolecular interaction energy difference in the dimer between quantum mechanical accuracy and molecular mechanical accuracy ΔE_{AB_inter}:
ΔE_{AB_inter}-E_{AB_inter_QM}-E_{AB_inter_MM}

E_{AB_inter_QM} is the intermolecular interaction energy in the dimer calculated with quantum mechanical accuracy which is calculated in step (3), and E_{AB_inter_MM} is the intermolecular interaction energy in the dimer calculated with classical mechanical accuracy.

Build up the interatomic distance matrices in the dimer set{D_{AB}}; build a convolutional neural network, wherein the interatomic distance matrix in the dimer as the input of the neural network, and the high-precision interaction correction of the dimer as the output; Use the interatomic distance matrix {M_{AB}} of this batch of dimers {D_{AB}} and the modified values {Σ_{AB_inter}} of their interaction energies to train the parameters of the neural network.

6) Calculate Crystal Energies

Calculate the total energy for any crystal structure S generated during the crystal prediction process:

$${E}_{S} = {\sum }_{a}^{mols} {E}_{a} + {\sum }_{AB}^{dimers} {E}_{AB\_MM} + {\sum }_{AB}^{dimers} Δ {E}_{AB\_inter} + \sum {E}_{others \_MM}$$

Eₐ^{mols} Eₐ is the sum of all intramolecular energies; Σ_{AB}^{dimers}E_{AB_MM} is the sum of all dimer energies calculated with classical mechanical accuracy, and Σ_{AB}^{dimers} ΔE_{AB_inter} is the sum of the correction amounts of the intermolecular interaction energy in all dimmers calculated by the neural network in step (5); ΣE_{others_MM} is all remaining interactions, calculated by conventional classical mechanics.

What is claimed is:

1. A double-layer neural network algorithm for high-precision energy calculation of organic molecular crystal structures, which includes the following steps:
(1) run a conventional crystal structure prediction
after energy ranking, determine a cut-off value of relative energy E₀; take out all crystal structures with relative energy lower than the cut-off value to get a set of crystal structures, and marked as {Sᵢ}, subscript i means to all crystal structures whose energies are lower than the cut-off value; calculate the energies of the structures in the set with quantum mechanical accuracy to obtain an accurate energy set marked as {Eᵢ};
(2) extract molecular conformations and calculate their energies
extract all molecular conformations from the crystal structure set {Sᵢ}, mark the molecular conformation set as {Cₐ}, subscript a means all molecular conformations that have occurred in all crystal structures; calculate the energies of the conformation in the set with quantum mechanical accuracy to get the accurate energies set as {Eₐ^{mol}};
(3) extract molecular dimers and calculate the intermolecular interaction energies
select a central unit cell for a crystal Sⱼ from the crystal structures set {Sᵢ}, and take a circle of molecules from all molecules in the central unit cell within their range of Van der Waals force; the range of Van der Waals force is defined as at least the distance between one pair atoms in two molecules is less than the sum of their Van der Waals radius plus 1.5 Å;
extract the central unit cell and all molecular dimers {D_{AB}} within Van der Waals force range, and calculate the intermolecular interaction energies in each dimer with quantum mechanical accuracy;
(4) build a convolutional neural network of single molecule conformational energy
mark the molecular flexible dihedral angle set as {Aₗ}, l means all the flexible dihedral angles in the molecule; set a series of fixed angle values as {θₛ}, for one of the angles Aₗ; conduct energy-constrained optimization calculations with the quantum mechanical accuracy to obtain a batch of molecular conformations and energies;
build a convolutional neural network, the atomic distance matrix Mₗ in the molecule is used as an input of the neural network, and the molecular conformational energies as an output; and use this batch of molecular conformations and the interatomic distance matrix of all the conformations obtained in step (2), and its conformation energies to train the parameters of the neural network;
(5) build a molecular dimer energy-corrected convolutional neural network
calculate the intermolecular interaction energies in all dimers obtained in step (3) with the classical mechanical accuracy; calculate the difference of intermolecular interaction energy in the dimer between the quantum mechanical accuracy and the molecular mechanical accuracy ΔE_{AB_inter};
build up an interatomic distance matrix of the dimer {D_{AB}}; build a convolutional neural network wherein the interatomic distance matrix in the dimer as the input of the neural network, and the high-precision interaction correction of the dimer as the output; use the interatomic distance matrix {M_{AB}} of the dimers {D_{AB}} and the modified values {ΔE_{AB_inter}} of their interaction energies to train the parameters of the neural network;
(6) calculate crystal energies
calculate the total energies for any crystal structure S generated during the crystal prediction process:
$${E}_{S} = {\sum }_{a}^{mols} {E}_{a} + {\sum }_{AB}^{dimers} {E}_{AB\_MM} + {\sum }_{AB}^{dimers} Δ {E}_{AB\_inter} + \sum {E}_{others \_MM}$$
here Σₐ^{mols} Eₐ is the sum of all intramolecular energies; Σ_{AB}^{dimers} E_{AB_MM} is the sum of all dimer energies calculated with classical mechanical accuracy, and Σ_{AB}^{dimers}ΔE_{AB_inter} is the sum of the correction amounts of the intermolecular interaction energies in all dimmers calculated by the neural network in step (5); ΣE_{others_MM} is all remaining interactions, calculated by conventional classical mechanics.

2. The double-layer neural network algorithm for high-precision energy calculation of organic molecular crystal structure according to claim 1, wherein calculate the intermolecular interaction energies of each dimer in step (3), in which the calculation formula is:
E_{AB_inter_QM}=E_{AB_tot_QM}−E_{A_QM}−E_{B_QM}
E_{AB_inter_QM} is the intermolecular interaction energy of dimer AB, E_{AB_inter_QM} is the total energy in the dimer, E_{A_QM} is the energy of the molecule A of the dimer; in the same way, E_{B_QM} is the energy of molecule B of the dimer, and all energy calculations are performed with quantum mechanical accuracy.

3. The double-layer neural network algorithm for high-precision energy calculation of organic molecular crystal structure according to claim 2, wherein calculate the difference between the quantum mechanical accuracy and molecular mechanical accuracy of the intermolecular interaction energy in the dimer in step (5), in which the calculation formula is:
ΔE_{AB_inter}=E_{AB_inter_QM}−E_{AB_inter_MM}
E_{AB_inter_QM} is the intermolecular interaction energy in the dimer calculated with quantum mechanical accuracy in step (3), E_{AB_inter_MM} is the intermolecular interaction energy of the dimer calculated with classic mechanical accuracy.
